(19) United States
(12) Patent Application Publication
(10) Pub. No.: US 2022/0207766 A1
(43) Pub. Date: Jun. 30, 2022

(54) DYNAMIC IMAGE ANALYSIS DEVICE, RECORDING MEDIUM, AND DYNAMIC IMAGE PROCESSING METHOD

(71) Applicant: KONICA MINOLTA, INC., Tokyo (JP)

(72) Inventor: Tatsuya TAKAGI, Tokyo (JP)

(73) Assignee: KONICA MINOLTA, INC., Tokyo (JP)

(21) Appl. No.: 17/562,089

(22) Filed: Dec. 27, 2021

(30) Foreign Application Priority Data
Dec. 28, 2020 (JP) 2020-218454

(51) Int. Cl. G06T 7/73; G06T 1/00

(52) U.S. Cl. CPC G06T 7/73; G06T 2207/20208; G06T 1/0007

(57) ABSTRACT

A dynamic image analysis device including a hardware processor that: acquires a dynamic image obtained from dynamic imaging by radiation; and performs a suitability judgement as to whether the dynamic image is suitable or unsuitable for a dynamic analysis based on the dynamic image or an analysis result obtained by analyzing the dynamic image.

### CROSS-REFERENCE TO RELATED APPLICATIONS

The entire disclosure of Japanese Patent Application No. 2020-218454 filed on Dec. 28, 2020 is incorporated herein by reference in its entirety.

### BACKGROUND

### Technological Field

The present invention relates to a dynamic image analysis device, a recording medium, and a dynamic image processing method.

### Description of the Related Art

Conventionally, a dynamic image obtained by radiography of a dynamic state having a periodicity of a subject has been used for diagnosis. With the dynamic image, it is possible to display and analyze the dynamic state of the subject, which was not captured by a still image.

In such a dynamic image, the reliability of the dynamic image affects the reliability of the image resulting from the analysis of the dynamic image, but it is not clear how reliable the image is just by looking at the image resulting from the analysis. Therefore, a dynamic image analysis device has been proposed that displays reliability information indicating the reliability of the dynamic image along with the image of the result of the dynamic image analysis in order to make the reliability of the image of the analysis result easier to understand. (See, for example, JP 2020-000807 A).

### SUMMARY

However, with the dynamic image analysis device described in the above-mentioned JP 2020-000807 A, although the reliability of the dynamic image analysis results can be ascertained from the reliability of the dynamic image, it is still a matter of subjective judgement by the technologist as to whether the results of the dynamic image analysis can be used for diagnosis.

The present invention has been made in consideration of the above matters, and an object of the present invention is to objectively determine whether the analysis results of dynamic image can be used for diagnosis.

To achieve at least one of the abovementioned objects, according to an aspect of the present invention, a dynamic image analysis device reflecting one aspect of the present invention includes a hardware processor that: acquires a dynamic image obtained from dynamic imaging by radiation; and performs a suitability judgement as to whether the dynamic image is suitable or unsuitable for a dynamic analysis based on the dynamic image or an analysis result obtained by analyzing the dynamic image.

To achieve at least one of the abovementioned objects, according to an aspect of the present invention, a dynamic image analysis device reflecting one aspect of the present invention includes a hardware processor that: acquires a dynamic image obtained from dynamic imaging by radiation; outputs unsuitability information when the dynamic image is unsuitable for a dynamic analysis; and performs control not to display, on a display, an analysis image obtained by analyzing a dynamic state of the dynamic image that is judged to be unsuitable for the dynamic analysis.

To achieve at least one of the abovementioned objects, according to an aspect of the present invention, a recording medium reflecting one aspect of the present invention is a non-transitory recording medium storing a computer readable program causing a computer to perform: acquiring that is acquiring a dynamic image obtained from dynamic imaging by radiation; and judging that is performing a suitability judgement of the dynamic image for a dynamic analysis based on the dynamic image or an analysis result obtained by analyzing the dynamic image.

To achieve at least one of the abovementioned objects, according to an aspect of the present invention, a recording medium reflecting one aspect of the present invention is a non-transitory recording medium storing a computer readable program causing a computer to perform: acquiring that is acquiring a dynamic image obtained from dynamic imaging by radiation; outputting that is outputting unsuitability information when the dynamic image is unsuitable for a dynamic analysis; and controlling that is controlling not to display, on a display, an analysis image obtained by analyzing a dynamic state of the dynamic image that is judged to be unsuitable.

To achieve at least one of the abovementioned objects, according to an aspect of the present invention, a dynamic image processing method reflecting one aspect of the present invention includes: acquiring a dynamic image that is obtained from dynamic imaging by radiation; and performing a suitability judgement of the dynamic image for a dynamic analysis based on the dynamic image or an analysis result obtained by analyzing the dynamic image.

To achieve at least one of the abovementioned objects, according to an aspect of the present invention, a dynamic image processing method reflecting one aspect of the present invention includes: acquiring a dynamic image that is obtained from dynamic imaging by radiation; outputting unsuitability information when the dynamic image is unsuitable for a dynamic analysis; and controlling not to display, on a display, an analysis image obtained by analyzing a dynamic state of the dynamic image that is judged to be unsuitable.

### BRIEF DESCRIPTION OF THE DRAWINGS

The advantages and features provided by one or more embodiments of the invention will become more fully understood from the detailed description given hereinafter and the appended drawings which are given by way of illustration only, and thus are not intended as a definition of the limits of the present invention, and wherein:

FIG. 1 is a diagram showing the overall configuration of a dynamic image analysis system according to an embodiment of the present invention;

FIG. 2 is a flowchart of imaging control processing executed by a controller of the imaging console shown in FIG. 1;

FIG. 3 is a flowchart of analysis availability judgement processing executed by a controller of the diagnostic console shown in FIG. 1 in the first embodiment;

FIG. 4 is an example of a judgement result screen displayed on a display of the diagnostic console shown in FIG. 1 in the first embodiment;

FIG. 5 is an example of a re-imaging necessity selection screen displayed on the display of the diagnostic console shown in FIG. 1 in the first embodiment;

FIG. 6 is an example of a judgement result screen displayed on the display of the diagnostic console shown in FIG. 1 in the first embodiment;

FIG. 7 is a flowchart of analysis result judgement processing executed by the controller of the diagnostic console shown in FIG. 1 in a second embodiment; and

FIG. 8 is an example of a judgement result screen displayed on the display of the diagnostic console shown in FIG. 1 in the second embodiment.

### DETAILED DESCRIPTION OF EMBODIMENTS

Embodiments of the present invention will be described below with reference to the accompanying drawings. However, the scope of the invention is not limited to the disclosed embodiments or the illustrated examples.

### First Embodiment

### Configuration of Dynamic Image Analysis System 100

First, the configuration of the first embodiment will be described.

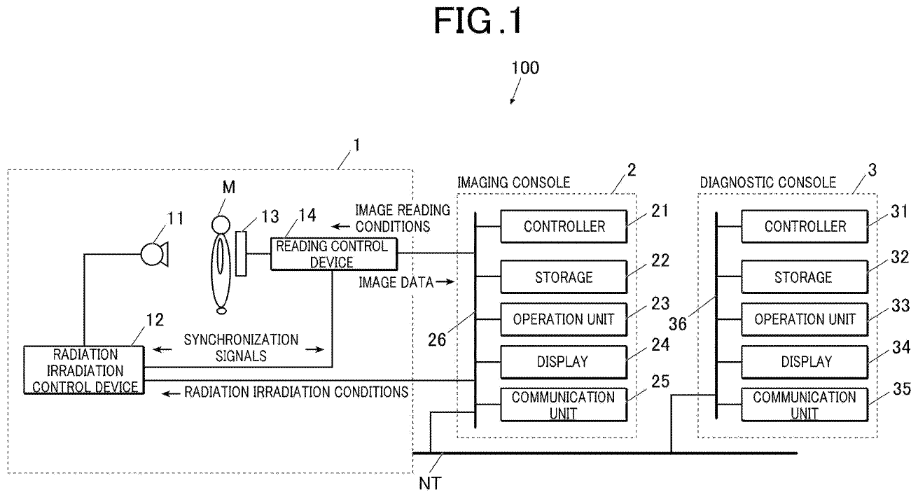

FIG. 1 shows the overall configuration of a dynamic image analysis system 100 according to the first embodiment.

As shown in FIG. 1, in the dynamic image analysis system 100, an imaging device 1 and an imaging console 2 are connected through a communication cable or the like, and the imaging console 2 and a diagnostic console 3 are connected through a communication network NT such as a local area network (LAN). The devices constituting the dynamic image analysis system 100 conform to the digital image and communications in medicine (DICOM) standard, and communication between the devices is performed in accordance with DICOM.

### Configuration of Imaging Device 1

The imaging device 1 is an imaging unit that images dynamic states having a periodicity (cycle), such as a change in the form of expansion and contraction of the lungs caused by respiratory movement, and heartbeat. Dynamic imaging refers to obtaining a plurality of images indicating the dynamic state of a subject by repeatedly emitting pulsed X-rays or other radiation to the subject at predetermined time intervals (pulse irradiation), or irradiating the subject continuously at a low dose rate (continuous irradiation). The dynamic imaging is imaging of a moving state or changing state, for recording a moving image. The dynamic imaging includes imaging of moving image, but does not include imaging of still image while displaying the moving image. A series of images obtained by dynamic imaging is referred to as a dynamic image. Each of the plurality of images constituting the dynamic image is referred to as a frame image. In the following embodiments, a case where dynamic imaging of a chest is performed by pulse irradiation will be described as an example.

A radiation source 11 is disposed in a position facing a radiation detector 13 with a subject M (examinee) interposed therebetween, and irradiates the subject M with radiation (X-rays) under the control by a radiation irradiation control device 12.

The radiation irradiation control device 12 is connected to the imaging console 2 and performs radiography, controlling the radiation source 11 according to the radiation irradiation conditions input from the imaging console 2. The radiation irradiation conditions input from the imaging console 2 include, for example, a pulse rate, a pulse width, a pulse interval, the number of imaging frames for one imaging operation, an X-ray tube current value, an X-ray tube voltage value, and the type of an additional filter. The pulse rate is the number of times of radiation irradiation per second, and matches the frame rate which will be described later. The pulse width is a radiation irradiation time for one radiation irradiation operation. The pulse interval is the time from the start of one radiation irradiation to the start of the next radiation irradiation, and matches a frame interval which will be described later.

The radiation detector 13 is a semiconductor image sensor such as an FPD. The FPD has a glass substrate, for example, and a plurality of detection elements (pixels) are arranged in a matrix in a predetermined position on the substrate. The detection elements detects radiation emitted from the radiation source 11 and passing through at least the subject M according to its intensity, and converts the detected radiation to an electric signal and accumulates it. Each pixel includes a switching unit such as a thin film transistor (TFT). The FPD is classified into the indirect conversion type FPD that converts X-rays into electric signals using a photoelectric conversion element via a scintillator, and the direct conversion type FPD that converts X-rays directly into electric signals, which are both applicable.

The radiation detector 13 faces the radiation source 11 with the subject M therebetween.

The reading control device 14 is connected to the imaging console 2. The reading control device 14 controls the switching unit of each pixel of the radiation detector 13 according to the image reading conditions, which are input from the imaging console 2, in order to make sequential switching for reading the electric signal accumulated in each pixel and thus read the electric signals accumulated in the radiation detector 13, thereby acquiring image data. This image data is a frame image. The reading control device 14 then outputs the acquired frame image to the imaging console 2. The image reading conditions include, for example, a frame rate, a frame interval, a pixel size, and an image size (matrix size). The frame rate is the number of frame images acquired per second, and matches the pulse rate. The frame interval is the time from the start of one operation for frame image acquisition to the start of the next operation for frame image acquisition, and matches the pulse interval.

In the embodiment, the radiation irradiation control device 12 and the reading control device 14 are connected to each other, and exchange synchronization signals so that their radiation irradiation operations and image reading operations can be synchronized.

### Configuration of Imaging Console 2

The imaging console 2 outputs radiation irradiation conditions and image reading conditions to the imaging device 1 to control radiography and radiographic image reading operation performed in the imaging device 1, and displays a dynamic image acquired through the imaging device 1, so that whether the image is suitable for checking or diagnosis of positioning by a radiographer, such as a radiation technologist, is confirmed.

As shown in FIG. 1, the imaging console 2 includes a controller 21, a storage 22, an operation unit 23, a display 24, and a communication unit 25, and they are connected via a bus 26

The controller 21 includes a central processing unit (CPU) and a random access memory (RAM). The CPU of the controller 21 reads, according to the operation given through the operation unit 23, system programs and various process programs stored in the storage 22 and expands them in the RAM, executes various processes, including an imaging control processing, which will be described later, according to the expanded programs, and centrally controls the operations of the components of the imaging console 2 and the radiation irradiating operation and reading operation in the imaging device 1.

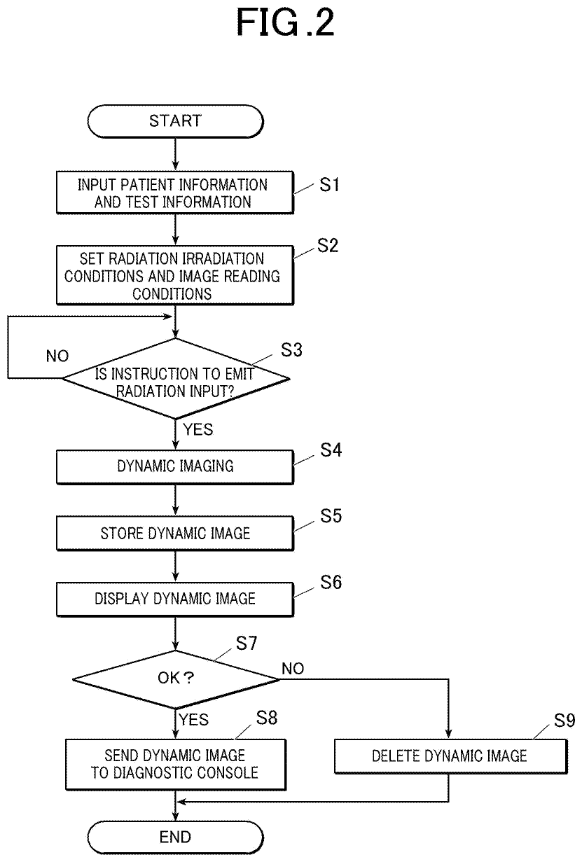

The storage 22 is a nonvolatile semiconductor memory, a hard disk, or the like. The storage 22 stores data such as various programs executed by the controller 21, parameters needed for execution of processes using programs, or processing results. For example, the storage 22 stores programs for executing the imaging control processing shown in FIG. 2. Further, the storage 22 stores the radiation irradiation conditions and the image reading conditions in association with the test target site (for example, the chest). These programs are stored in the form of computer-readable program codes, and the controller 21 sequentially executes operations according to the program codes.

The operation unit 23 includes a keyboard having cursor keys, numeric keys, various function keys, and the like, and a pointing device such as a mouse, and outputs an instruction signal, which is input through a key operation on the keyboard or a mouse operation, to the controller 21. The operation unit 23 may include a touch panel on the screen of the display 24. In this case, the operation unit 23 outputs an instruction signal, which is input via the touch panel, to the controller 21.

The display 24 includes a monitor, such as a liquid crystal display (LCD) or a cathode ray tube (CRT), and displays an input instruction from the operation unit 23, data, and the like according to an instruction indicated by a display signal input from the controller 21.

The communication unit 25 includes a LAN adapter, a modem, and a terminal adapter (TA), and controls data exchange with the devices connected to the communication network NT.

### Configuration of Diagnostic Console 3

The diagnostic console 3 is a device for acquiring dynamic images from the imaging console 2 and displaying the acquired dynamic images and the analysis results (analysis images) of the dynamic images, to support a doctor's diagnosis.

As shown in FIG. 1, the diagnostic console 3 includes a controller 31 (hardware processor), a storage 32, an operation unit 33, a display 34, and a communication unit 35, and they are connected via a bus 36.

The controller 31 includes a CPU and a RAM. The CPU of the controller 31 reads, according to the operation given through the operation unit 33, system programs and various process programs stored in the storage 32 and expands them in the RAM, executes various processes, including analysis availability judgement processing, which will be described later, according to the expanded programs, and centrally controls the operations of the components of the diagnostic console 3.

The storage 32 is a nonvolatile semiconductor memory, a hard disk, or the like. The storage 32 stores data such as various programs, including a program for executing the analysis availability judgement processing in the controller 31, parameters needed for execution of processes using programs, or processing results. These various programs are stored in the form of computer-readable program codes, and the controller 31 sequentially executes operations according to the program codes.

In the storage 32, dynamic images captured in the past are stored in association with patient information (for example, patient ID, patient name, height, weight, age, and gender), test information (for example, test ID, test date, test target site), and the type of dynamic state of the diagnosis target (for example, resting breathing, deep breathing, breath holding, etc.) included in test order (order information). The electronic medical record information corresponding to the dynamic image may be acquired from an electronic medical record device not shown in the drawings and stored in association with the dynamic image.

The operation unit 33 includes a keyboard having cursor keys, numeric keys, various function keys, and the like, and a pointing device such as a mouse, and outputs an instruction signal, which is input through a user's key operation on the keyboard or a user's mouse operation, to the controller 31. The operation unit 33 may include a touch panel on the display screen of the display 34. In this case, the operation unit 33 outputs an instruction signal, which is input via the touch panel, to the controller 31.

The display 34 is a monitor, such as an LCD or a CRT, and performs various types of displaying according to an instruction indicated by a display signal input from the controller 31.

The communication unit 35 includes a LAN adapter, a modem, and a TA, and controls data exchange with the devices connected to the communication network NT.

### Operation of Dynamic Image Analysis System 100

The operation of the dynamic image analysis system 100 according to this embodiment will now be described. cl Operations of Imaging Device 1 and Imaging Console 2

The imaging operation by the imaging device 1 and the imaging console 2 will be first described.

FIG. 2 shows an imaging control processing executed in the controller 21 of the imaging console 2. The imaging control processing is executed by cooperation of the controller 21 and a program stored in the storage 22.

First, the operation unit 23 of the imaging console 2 is operated by the radiographer to input patient information and test information of the subject (Step S1).

Next, the radiation irradiation conditions are read from the storage 22 and set in the radiation irradiation control device 12, and the image reading conditions are read from the storage 22 and set in the reading control device 14 (Step S2).

Next, an instruction to emit radiation by the operation on the operation unit 23 is put on standby (Step S3). Here, the radiographer performs positioning by locating the subject M between the radiation source 11 and the radiation detector 13. Further, an instruction related to a respiratory state according to the type of dynamic state of the diagnosis target is given to the subject M. Upon completion of preparation for imaging, the operation unit 23 is operated to input an instruction to emit radiation.

If the instruction to emit radiation is input from the operation unit 23 (Step S3; YES), an instruction to start imaging is output to the radiation irradiation control device 12 and the reading control device 14, and dynamic imaging is started (Step S4). In other words, radiation is emitted from the radiation source 11 at pulse intervals set in the radiation irradiation control device 12, and a frame image is acquired by the radiation detector 13.

After a predetermined number of frames are captured, the controller 21 outputs an instruction to end the imaging to the radiation irradiation control device 12 and the reading control device 14, thereby stopping the imaging operation. The number of captured frames is the number of images with which at least one respiratory cycle can be captured.

The frame images obtained by the imaging are sequentially input to the imaging console 2 and stored in the storage 22 in association with numbers indicating the imaging order (frame numbers) (Step S5), and displayed on the display 24 (Step S6). The radiographer checks the positioning or the like according to the displayed dynamic image, and judges whether an image suitable for diagnosis has been obtained by the imaging (imaging OK) or whether re-imaging is necessary (imaging NG). The operation unit 23 is then operated to input a judgement result.

If the judgement result indicating imaging OK is input through a predetermined operation on the operation unit 23 (Step S7; YES), an identification ID for identifying a dynamic image, patient information, test information, radiation irradiation conditions, image reading conditions, a number indicating the imaging order (frame number), and other information included in the order information are assigned to each of a series of frame images acquired by dynamic imaging (for example, written, in DICOM format, to the header region of image data), and sent to the diagnostic console 3 via the communication unit 25 (Step S8). This processing then ends. In contrast, when the judgement result indicating imaging NG is input through a predetermined operation on the operation unit 23 (Step S7; NO), the series of frame images stored in the storage 22 is deleted (Step S9), and this processing then ends. In this case, re-imaging is required.

### Operation of Diagnostic Console 3

Next, the operation of the diagnostic console 3 will be described.

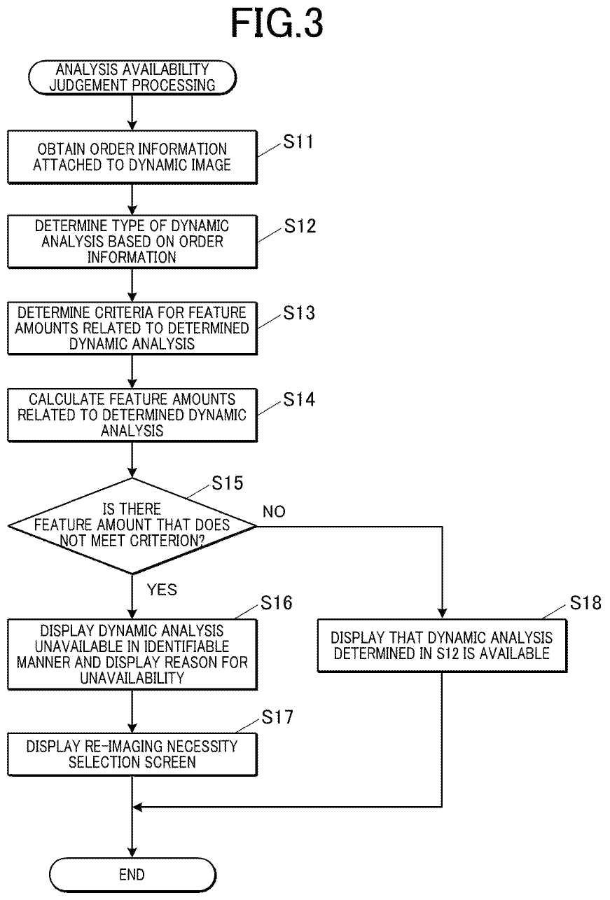

In the diagnostic console 3, upon reception of a series of frame images of the dynamic image from the imaging console 2 via the communication unit 35, analysis availability judgement processing shown in FIG. 3 is performed through the cooperation between the controller 31 and the program stored in the storage 32.

When the analysis availability judgement processing is started, the controller 31 of the diagnostic console 3 first obtains the order information attached to the dynamic image received via the communication unit 35 (Step S11).

Next, the controller 31 determines the type of dynamic analysis based on the order information obtained in step S11 (step S12). Specifically, the controller 31 determines the type of dynamic analysis based on the type of dynamic state to be diagnosed (e.g., resting breathing, deep breathing, breath holding, etc.) included in the order information. The dynamic analysis in this embodiment means analyzing the dynamic image for the purpose of providing data to be used for reference of diagnosis by the doctor. As the type of dynamic analysis, there are, for example, ventilation analysis, blood flow analysis, maximum lung field area, lung field area change rate, diaphragmatic movement amount, lung field movement analysis, etc. In addition to these types of analyses, there is adhesion analysis, which analyzes whether or not tissues that should be separated from each other are attached, and orthopedic analysis, which measures the angle and distance between bones in a joint.

More specifically, in the storage 32, the type of dynamic analysis is stored in association with each type of dynamic state to be diagnosed. For example, for the dynamic state type “breath holding,” two types of dynamic analyses are stored: blood flow analysis and maximum lung field area. For example, for the dynamic state type “deep breathing,” four types of dynamic analyses are stored: ventilation analysis, lung field area change rate, diaphragmatic movement amount, and lung field movement analysis.

This means that if the type of dynamic state to be diagnosed included in the order information acquired in step S11 is “breath holding,” two types of dynamic analyses corresponding to this “breath holding” are determined: blood flow analysis and maximum lung field area.

If the type of dynamic state to be diagnosed included in the order information obtained in step S11 is “deep breathing,” four types of dynamic analyses are determined for this “deep breathing”: ventilation analysis, lung field area change rate, diaphragmatic movement amount, and lung field movement analysis.

Next, the controller 31 determines the criteria for the feature amounts related to the dynamic analysis determined in step S12 (Step S13).

Specifically, the storage 32 stores the criteria of relevant feature amounts for each type of dynamic analysis. For example, for blood flow analysis, the following (1-1) to (1-5) criteria are stored in association with blood flow analysis, as criteria of feature amounts.

(1-1) S value (dose index)<5000
(1-2) Diaphragmatic movement amount≤5 mm
(1-3) Body movement≤10 mm
(1-4) Accuracy of decubitus position≥90%
(1-5) Accuracy of not missing lung field≥90

For example, the following criteria (2-1) to (2-2) are stored in association with the maximum lung field area, as the criteria for feature amounts.

(2-1) Accuracy of being maximal inspiration≥90%
(2-2) Accuracy of not missing lung field≥90%

For example, for ventilation analysis, the following criteria (3-1) to (3-6) are stored in association with ventilation analysis, as the criteria for feature amounts.

(3-1) S value<5000
(3-2) Diaphragmatic movement amount≥5 mm
(3-3) Body movement≤10 mm
(3-4) Accuracy of not missing lung field≥90%
(3-5) Difference of respiratory cycle from the past dynamic image≤10%
(3-6) Accuracy of decubitus position≥90%

For example, the following criteria (4-1) to (4-2) are stored in association with the rate of change of lung field area, as the criteria for feature amounts.

(4-1) Accuracy of not missing lung field≥90%
(4-2) Accuracy of including maximal inspiration≥90%

For example, the following criteria (5-1) to (5-2) are stored in association with the diaphragmatic movement amount, as the criteria for the feature amounts.

(5-1) Body movement≤5 mm
(5-2) Accuracy of not missing diaphragm≥90%

For example, the following criteria (6-1) to (6-3) are stored in association with the movement analysis of lung field, as the criteria for the feature amounts.

(6-1) Accuracy of not missing lung field≥90%
(6-2) Accuracy of the decubitus position≥90%
(6-3) Diaphragmatic movement amount≥5 mm

In this way, when the dynamic analysis determined in step S12 is the two types of dynamic analyses that are blood flow analysis and maximum lung field area, the criteria for the feature amounts related to each of blood flow analysis and maximum lung field area are determined based on the above criteria stored in storage 32.

In addition, when the dynamic analysis determined in step S12 is the four types of ventilation analysis, lung field area change rate, diaphragmatic movement amount, and lung field movement analysis, the criteria for the feature amounts related to each of ventilation analysis, lung field area change rate, diaphragmatic movement amount, and lung field movement analysis are determined based on the above-mentioned criteria stored in the storage 32.

Next, the controller 31 calculates the feature amounts related to the dynamic analysis determined in step S12 (Step S14).

For example, if the dynamic analysis determined in step S12 is the two types of blood flow analysis and maximum lung field area, the controller 31 calculates the S value, diaphragmatic movement amount, body movement, accuracy of decubitus position, accuracy of not missing lung field, and accuracy of being maximal inspiration as the feature amounts respectively based on the dynamic image received from imaging console 2.

For example, if the dynamic analysis determined in step S12 is the four types of ventilation analysis, lung field area change rate, diaphragmatic movement amount, and lung field movement analysis, the controller 31 calculates, as the feature amounts, the S value, diaphragmatic movement amount, body movement, accuracy of decubitus position, accuracy of not missing lung field, difference of respiratory cycle from the past dynamic image, accuracy of including maximal inspiration, and accuracy of not missing diaphragm respectively based on the dynamic image received from imaging console 2.

Next, the controller 31 determines whether there is a feature amount that does not meet the criterion thereof determined in step S13 (Step S15).

If, in step S15, it is determined that there is a feature amount that does not meet the criterion thereof determined in step S13 (Step S15; YES), the controller 31 displays on the display 34 a judgement result screen that identifiably displays the fact that the dynamic analysis for which the criterion for the feature amounts was determined is not available (cannot be performed) (unsuitability information) and also displays the reason why the dynamic analysis is not available (unsuitability information) (Step S16).

For example, in the case where the dynamic analysis determined in step S12 is the two types of blood flow analysis and maximum lung field area and the controller 31 calculates the S value (e.g., 3000), diaphragmatic movement amount (e.g., 8 mm), body movement (e.g., 5 mm), accuracy of decubitus position (e.g., 90%), accuracy of not missing lung field (e.g., 99.9%), and accuracy of being maximal inspiration (e.g., 90%) as the feature amounts respectively based on the dynamic image received from imaging console 2, the above diaphragmatic movement amount (e.g., 8 mm) is determined as the feature amounts that does not meet the criterion (diaphragmatic movement amount≤5 mm). Accordingly, the controller 31 displays on the display 34 a judgement result screen 41 that identifiably displays the fact that the blood flow analysis for which the criterion for the feature amounts was determined to be “diaphragmatic movement amount≤5 mm” is not possible (not available) and also displays the reason why the blood flow analysis is not possible. In such a case, the fact that the maximum lung field area can be analyzed is displayed on the judgement result screen 41 because all the feature amounts related to the maximum lung field area meet the criteria. The controller 31 may, for example, output an audio message that the blood flow analysis is not possible and also outputs an audio message regarding the reason why the analysis cannot be performed, in conjunction with the display (output) of the judgement result screen 41 on the display 34. In addition, the controller 31 may, for example, perform a decorative light-emitting display such as blinking a list display region 411 described below when displaying the judgement result screen 41 on the display 34.

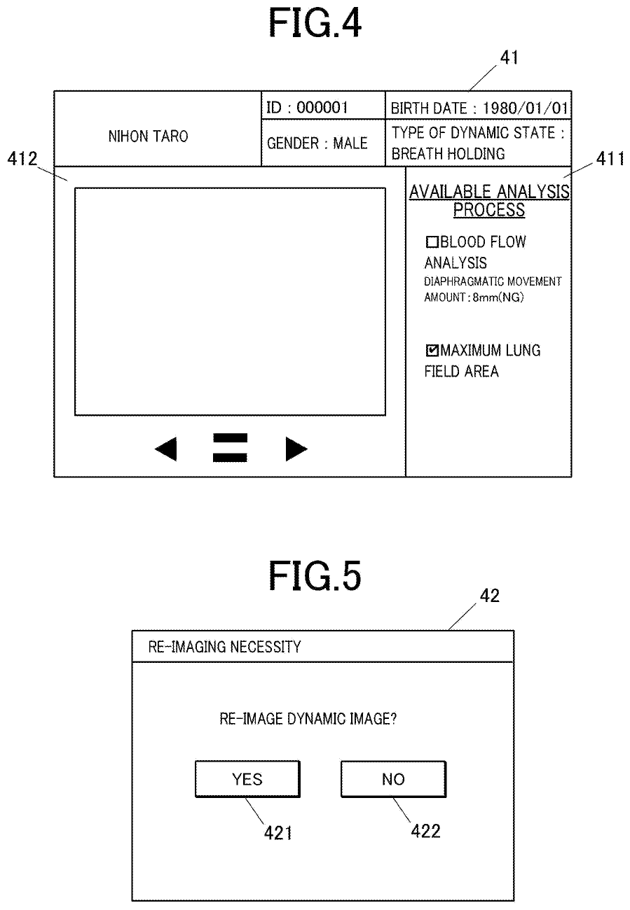

FIG. 4 shows an example of the above judgement result screen 41. In this judgement result screen 41, a list display region 411 is provided on the right side of the screen to display a list of available analysis processes.

In this list display region 411, the fact that blood flow analysis cannot be performed is displayed in an identifiable manner, i.e., the check box corresponding to blood flow analysis is unchecked. In addition, the reason why blood flow analysis is not available (e.g., diaphragmatic movement amount: 8 mm (NG)) is displayed. On the other hand, the fact that the maximum lung field area can be analyzed is displayed in an identifiable manner, i.e., a check box corresponding to the maximum lung field area is checked.

In addition, in the judgement result screen 41, an analysis image display region 412 is provided on the left of the list display region 411, and the analysis image of dynamic analysis that is judged to be available can be displayed in the analysis image display region 412.

In the above example, since the maximum lung field area is judged to be analyzable, the analysis image regarding this maximum lung field area can be displayed in the analysis image display region 412. On the other hand, in the above example, since blood flow analysis is judged to be unavailable, control processing is performed so that the analysis image of this blood flow analysis is not displayed in the analysis image display region 412.

Returning to the description of the analysis availability judgement processing, the controller 31 displays the above judgement result screen 41 on the display 34, then pops up a re-imaging necessity selection screen 42 on the display 34 to allow the user to select whether or not to perform re-imaging of the dynamic image (Step S17), and ends the analysis availability judgement processing.

FIG. 5 shows an example of the re-imaging necessity selection screen 42 described above. In this re-imaging necessity selection screen 42, a message (message information) asking the user whether or not to re-image the dynamic image (e.g., character information of “Re-image dynamic image?”) is displayed, and YES button 421 and NO button 422 are provided.

The YES button 421 is a button for responding YES to the above message information. When the YES button 421 is selected via the operation unit 33, the controller 31 sends information that re-imaging of the dynamic image is required to the imaging console 2 via the communication unit 35. When the YES button 421 is selected, the display of the re-imaging necessity selection screen 42 is hidden.

The NO button 422 is a button for giving a NO response to the above message information. When the NO button 422 is selected via the operation unit 33, the display of the re-imaging necessity selection screen 42 is hidden.

In step S15, if it is determined that there is no feature amount that does not meet the criterion thereof determined in step S13 (Step S15; NO), the controller 31 displays the judgement result screen on the display 34 indicating that the dynamic analysis determined in step S12 is available (can be performed) (Step S18), and ends the analysis availability judgement processing.

For example, if the dynamic analysis determined in step S12 is the two types of blood flow analysis and maximum lung field area, and based on the dynamic image received from the imaging console 2, the S value (e.g., 3000), diaphragmatic movement amount (e.g., 5 mm), body movement (e.g., 5 mm), accuracy of decubitus position (e.g., 90%), accuracy of not missing lung field (e.g., 99.9%), and accuracy of being maximal inspiration (e.g., 90%) are calculated as the feature amounts, it is determined that there is no feature amounts that does not meet the criteria of the feature amounts determined in step S13. Therefore, the controller 31 displays the judgement result screen 43 on the display 34, which indicates that the blood flow analysis and the maximum lung field area are analyzable.

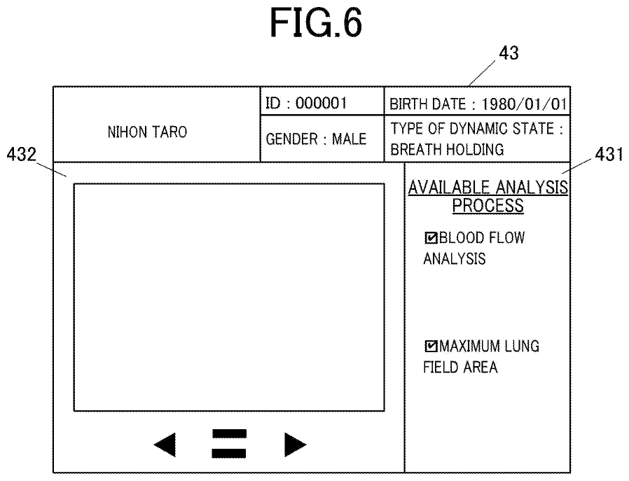

FIG. 6 shows an example of the above judgement result screen 43. In this judgement result screen 43, a list display region 431 is provided on the right side of the screen to display a list of available analysis processes, similarly to the judgement result screen 41 described above.

In this list display region 431, it is indicated that blood flow analysis and maximum lung field area can be analyzed, i.e., the respective checkboxes corresponding to blood flow analysis and maximum lung field area are checked.

In the judgement result screen 43, an analysis image display region 432 is provided to the left of the list display region 431, as in the judgement result screen 41 described above. The analysis image of the dynamic analysis that is judged to be available can be displayed in the analysis image display region 432.

In the example above, since the blood flow analysis and maximum lung field area are judged to be analyzable, the respective analysis images regarding the blood flow analysis and maximum lung field area can be displayed in the analysis image display region 432.

As described above, the diagnostic console 3 of the first embodiment acquires the dynamic image obtained from the dynamic imaging by radiation, and performs suitability judgement as to whether the dynamic image is suitable or unsuitable for the dynamic analysis based on the dynamic image.

Therefore, according to the diagnostic console 3 of the first embodiment, it is possible to objectively judge whether the analysis result of the dynamic image is usable for diagnosis by performing suitability judgement as to whether the dynamic image is suitable or unsuitable for the dynamic analysis based on the acquired dynamic image.

The diagnostic console 3 in the first embodiment judges whether the dynamic image is suitable or unsuitable for the dynamic analysis on the basis of the feature amounts related to the dynamic analysis obtained from the dynamic image.

Therefore, according to the diagnostic console 3 of the first embodiment, suitability judgement can be accurately performed by performing the suitability judgement based on the feature amounts related to the dynamic analysis obtained from the dynamic image.

In addition, the diagnostic console 3 of the first embodiment has determined the type of dynamic analysis to perform suitability judgement from among multiple types of dynamic analyses.

Therefore, according to the diagnostic console 3 of the first embodiment, the suitability judgement can be efficiently performed since the dynamic analysis to perform suitability judgement can be selected.

In addition, the diagnostic console 3 of the first embodiment has determined the type of dynamic analysis to perform suitability judgement on the basis of the order information.

Therefore, according to the diagnostic console 3 of the first embodiment, the suitability judgement can be efficiently and appropriately performed since the dynamic analysis to perform suitability judgement can be selected appropriately according to the order information.

Also, in the diagnostic console 3 of the first embodiment, the result of suitability judgement is displayed on the display 34.

Therefore, according to the diagnostic console 3 of the first embodiment, by displaying the result of the suitability judgement on the display 34, the user is able to grasp the result of the suitability judgement.

In addition, the diagnostic console 3 of the first embodiment does not display the analysis image of the dynamic image that is judged to be unsuitable by the suitability judgement on the display 34.

Therefore, the diagnostic console 3 of the first embodiment can prevent the analysis image of the dynamic image that is judged to be unsuitable by suitability judgement from being used for diagnosis.

In addition, in the diagnostic console 3 of the first embodiment, the analysis image of the dynamic image that is judged to be suitable by suitability judgement is displayed on the display 34.

Therefore, according to the diagnostic console 3 of the first embodiment, only the analysis image of dynamic image that is judged to be suitable by suitability judgement is used for diagnosis, and thus diagnosis using analysis image of dynamic image can be performed appropriately.

In addition, in the diagnostic console 3 of the first embodiment, when suitability judgement judging the dynamic image to be unsuitable is made, notification regarding re-imaging is made.

Therefore, according to the diagnostic console 3 of the first embodiment, when the dynamic image is judged to be unsuitable, it is possible to urge re-imaging of the dynamic image by notification regarding re-imaging, and therefore, a series of work regarding dynamic analysis can be performed smoothly.

In the diagnostic console 3 of the first embodiment, the dynamic image obtained from dynamic imaging by radiation is acquired, and if the dynamic image is unsuitable for dynamic analysis, the fact that dynamic analysis is not possible (available) and the reason why it is not possible (unsuitability information) are output, and the diagnostic console 3 does not display the analysis image obtained by dynamic analysis of the dynamic image judged to be unsuitable on the display 34.

Therefore, according to the diagnostic console 3 of the first embodiment, by outputting the fact that the dynamic analysis is not possible and the reason why the analysis is not possible (unsuitability information), the diagnostic console 3 can make the user grasp that the dynamic image is not suitable for the dynamic analysis. In addition, by not displaying on the display 34 the analysis image obtained by dynamic analysis of the dynamic image that is judged to be unsuitable, it is possible to prevent the said analysis image from being used for diagnosis.

In the diagnostic console 3 of the first embodiment, if a dynamic image is suitable for dynamic analysis, the analysis image obtained by dynamic analysis of the dynamic image that is judged to be suitable is displayed on the display 34.

Therefore, according to the diagnostic console 3 of the first embodiment, only the analysis image obtained by dynamic analysis of the dynamic image that is judged to be suitable for the dynamic analysis is used for diagnosis, so that diagnosis using the analysis image of the dynamic image can be performed appropriately.

### Second Embodiment

The second embodiment of the present invention is described below.

Different from the first embodiment described above, in the second embodiment, after dynamic analysis is performed on the dynamic image in the diagnostic console 3, suitability judgement is performed on the dynamic image based on the result of the dynamic analysis, to judge whether or not it is suitable for dynamic analysis.

The configuration in the second embodiment is the same as that described in the first embodiment except that the program for executing the analysis result judgement processing is stored in the storage 32 of diagnostic console 3, so the description is omitted. Hereinafter, the operation of the second embodiment will be described.

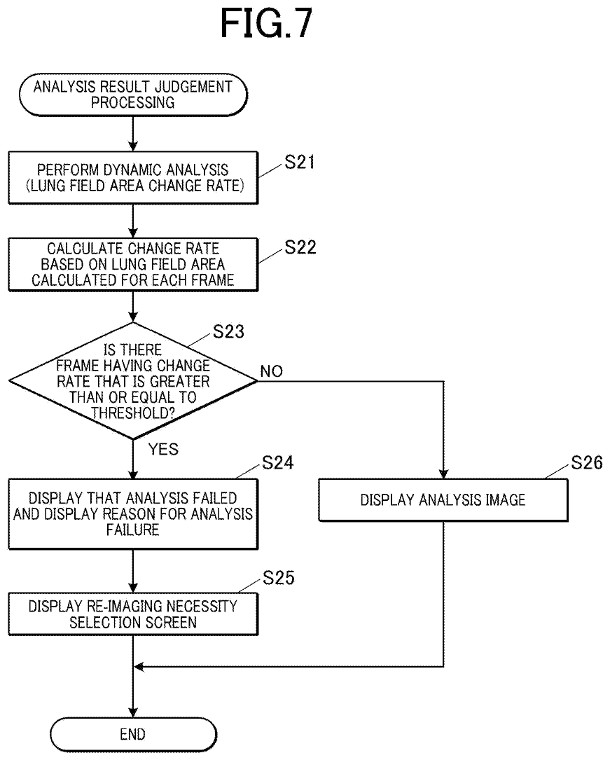

FIG. 7 is a flowchart showing the analysis result judgement processing executed by diagnostic console 3 in the second embodiment. This analysis result judgement processing is performed in cooperation with the controller 31 and the program stored in the storage 32, triggered by the reception of a series of frame images of dynamic image from the imaging console 2 via the communication unit 35.

When the analysis result judgement processing is started, the controller 31 of the diagnostic console 3 first performs the dynamic analysis (e.g., lung field area change rate), triggered by an input operation that instructs the controller 31 to perform the dynamic analysis via the operation unit 33 (Step S21).

Next, the controller 31 calculates the change rate (second feature amount) of the lung field area based on the lung field area calculated for each frame of the dynamic image (Step S22).

The controller 31 judges whether there is a frame having the change rate of the lung field area that is greater than or equal to the threshold (Step S23).

In step S23, if it is judged that there is a frame having the change rate of the lung field area that is greater than or equal to the threshold (step S23; YES), the controller 31 displays on the display 34 a judgement result screen 44 which displays that the analysis has failed and displays the reason for analysis failure (Step S24).

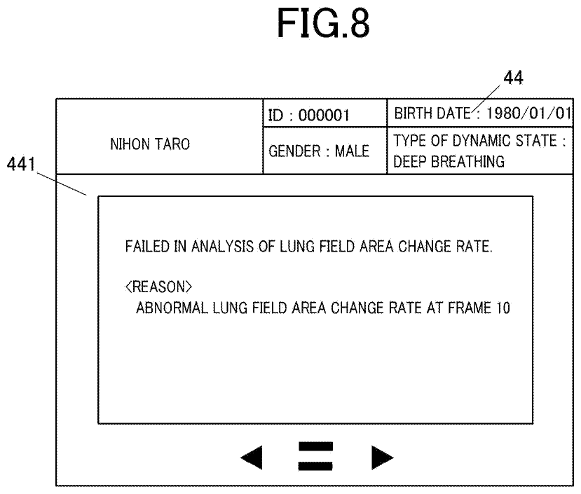

FIG. 8 shows an example of the above judgement result screen 44. The judgement result screen 44 has an analysis image display region 441 in the center of the screen, and message information indicating that the analysis failed (e.g., character information of “Failed in analysis of lung field area change rate.”) is displayed in this analysis image display region 441, as well as the reason for the analysis failure (e.g., character information of “Abnormal lung field area change rate at frame 10”). In other words, if the dynamic analysis fails, control is performed so that the analysis image for the dynamic analysis is not displayed in the analysis image display region 441. The controller 31 may, for example, output an audio message of message information that the analysis has failed and of the reason why the analysis has failed in conjunction with the display (output) of the judgement result screen 44 on the display 34. In addition, the controller 31 may, for example, perform a decorative light-emitting display such as blinking analysis image display region 441 when displaying the judgement result screen 44 on the display 34.

After displaying the judgement result screen 44 on the display 34, the controller 31 pops up on the display 34 the re-imaging necessity selection screen 42 (see FIG. 5) making the user select whether to perform re-imaging of the dynamic image (Step S25), and ends the analysis result judgement processing.

In step S23, if it is determined that there is no frame having the change rate of the lug filed area that is greater than or equal to the threshold (Step S23; NO), that is, if it is determined that the analysis of the lung field area change rate has been appropriately performed, the controller 31 displays the analysis image (not shown in the drawings) of the lung filed area change rate in the analysis image display region 441 of the judgement result screen 44 (see FIG. 8) (Step S26), and ends the analysis result judgement processing.

As described above, the diagnostic console 3 according to the second embodiment acquires the dynamic image obtained from the dynamic imaging with radiation, and performs the suitability judgement as to whether the dynamic image is suitable or unsuitable for the dynamic analysis on the basis of the analysis result obtained by analyzing the dynamic image.

Accordingly, according to the diagnostic console 3 in the second embodiment, it is possible to objectively determine whether the analysis result is usable for diagnosis by performing the suitability judgement as to whether the dynamic image is suitable or unsuitable for the dynamic analysis on the basis of the analysis result obtained by analyzing the dynamic image.

The diagnostic console 3 in the second embodiment performs the suitability judgement on the basis of the second feature amounts (ex. change rate of lung field area) regarding the dynamic analysis obtained from the analysis result.

Therefore, according to the diagnostic console 3 of the second embodiment, suitability judgement can be accurately performed by performing the suitability judgement based on the second feature amounts (ex. change rate of lung field area) regarding the dynamic analysis obtained from the analysis result.

Also, in the diagnostic console 3 of the second embodiment, the result of suitability judgement is displayed on the display 34.

Therefore, according to the diagnostic console 3 of the second embodiment, by displaying the result of the suitability judgement on the display 34, the user is able to grasp the result of the suitability judgement.

In addition, the diagnostic console 3 of the second embodiment does not display the analysis image of the dynamic image that is judged to be unsuitable by the suitability judgement on the display 34.

Therefore, the diagnostic console 3 of the second embodiment can prevent the analysis image of the dynamic image that is judged to be unsuitable by suitability judgement from being used for diagnosis.

In the diagnostic console 3 of the second embodiment, the analysis image of the dynamic image that is judged to be suitable by suitability judgement is displayed on the display 34.

Therefore, according to the diagnostic console 3 of the second embodiment, only the analysis image of dynamic image that is judged to be suitable by suitability judgement is used for diagnosis, and thus diagnosis using analysis image of dynamic image can be performed appropriately.

In the diagnostic console 3 of the second embodiment, when suitability judgement judging the dynamic image to be unsuitable is made, notification regarding re-imaging is made.

Therefore, according to the diagnostic console 3 of the second embodiment, when the dynamic image is judged to be unsuitable, it is possible to urge re-imaging of the dynamic image by notification regarding re-imaging, and therefore, a series of work regarding dynamic analysis can be performed smoothly.

Though the first embodiment and the second embodiment of the present invention have been described above, the described contents in the embodiments are preferred examples of the present invention, and the present invention is not limited to the above examples.

For example, in the first embodiment, if it is determined that there is a feature amount that does not meet the criterion thereof determined in step S13, in step S15 of the analysis availability judgement processing (see FIG. 3) (Step S15; YES), the controller 31 of the diagnostic console 3 displays on the display 34 the judgement result screen that displays in an identifiable manner that the dynamic analysis for which the criteria of the feature amounts has been determined is not possible (available) and that displays the reason why the dynamic analysis is not possible. In the case where none of the dynamic analyses determined in step S12 is possible, the controller 31 may delete the dynamic image acquired from the imaging console 2 (dynamic image which is the target of dynamic analysis).

The second embodiment has been described by taking, as an example, a case where the controller 31 of the diagnostic console 3 executes the analysis result judgement processing (see FIG. 7). However, the controller 31 may first execute the analysis availability judgement processing (see FIG. 3) described in the first embodiment, and then execute the analysis result judgement processing described in the second embodiment to the dynamic analysis which has been determined to be possible in the analysis availability judgement processing.

In the first embodiment, the type of dynamic analysis is determined on the basis of the order information in step S12 of the analysis availability judgement processing (see FIG. 3). However, the type of dynamic analysis desired by the user may be selected manually by, for example, the user performing a predetermined operation via the operation unit 33, not on the basis of the order information.

In the first embodiment and the second embodiment, the diagnostic console 3 has been described as an example of the dynamic image analysis device according to the present invention. However, the dynamic image analysis device may be a PC (Personal Computer) that specializes in dynamic analysis, an image detecting terminal, and the like.

In the second embodiment, the lung field area change rate is described as an example as the dynamic analysis which is the target of the analysis result judgement processing (see FIG. 7) in this processing, and the suitability judgement is performed on the basis of the lung field area change rate (second feature amount). However, for example, when the blood flow analysis is performed, the controller 31 may perform the suitability judgement on the basis of the heart ROI (second feature amounts) regarding the blood flow analysis.

In addition, for example, the above description discloses an example of using a hard disk, a semiconductor nonvolatile memory and the like as the computer readable medium of the program according to the present invention. However the present invention is not limited to the example. A portable recording medium such as a CD-ROM can be applied as other computer readable medium. A carrier wave is also applied as a medium providing the program data according to the present invention via a communication line.

As for the other detailed configurations and detailed operations of the devices forming the dynamic image analysis system, modifications can be made as needed within the scope of the present invention.

Although embodiments of the present invention have been described and illustrated in detail, the disclosed embodiments are made for purposes of illustration and example only and not limitation. The scope of the present invention should be interpreted by terms of the appended claims.

What is claimed is:

1. A dynamic image analysis device comprising a hardware processor that:
acquires a dynamic image obtained from dynamic imaging by radiation; and
performs a suitability judgement as to whether the dynamic image is suitable or unsuitable for a dynamic analysis based on the dynamic image or an analysis result obtained by analyzing the dynamic image.

2. The dynamic image analysis device according to claim 1, wherein the hardware processor performs the suitability judgement based on a feature amount related to the dynamic analysis, the feature amount being obtained from the dynamic image.

3. The dynamic image analysis device according to claim 1, wherein the hardware processor performs the suitability judgement based on a second feature amount regarding the dynamic analysis, the second feature amount being obtained from the analysis result.

4. The dynamic image analysis device according to claim 1, wherein
the dynamic analysis includes multiple types of dynamic analyses; and
the hardware processor determines a type of a dynamic analysis for which the suitability judgement is to be performed, from among the multiple types of the dynamic analyses.

5. The dynamic image analysis device according to claim 4, wherein the hardware processor determines the type of the dynamic analysis for which the suitability judgement is to be performed, based on order information.

6. The dynamic image analysis device according to claim 1, wherein the hardware processor performs control to display a result of the suitability judgement on a display.

7. The dynamic image analysis device according to claim 6, wherein the hardware processor performs control not to display, on the display, an analysis image of the dynamic image that is judged to be unsuitable by the suitability judgement.

8. The dynamic image analysis device according to claim 6, wherein the hardware processor displays, on the display, an analysis image of the dynamic image that is judged to be suitable by the suitability judgement.

9. The dynamic image analysis device according to claim 1, wherein the hardware processor performs notification regarding re-imaging when the dynamic image is judged to be unsuitable for the dynamic analysis.

10. A dynamic image analysis device comprising a hardware processor that:
acquires a dynamic image obtained from dynamic imaging by radiation;
outputs unsuitability information when the dynamic image is unsuitable for a dynamic analysis; and
performs control not to display, on a display, an analysis image obtained by analyzing a dynamic state of the dynamic image that is judged to be unsuitable for the dynamic analysis.

11. The dynamic image analysis device according to claim 10, wherein the hardware processor performs notification regarding re-imaging when the dynamic image is judged to be unsuitable for the dynamic analysis.

12. The dynamic image analysis device according to claim 10, wherein, when the dynamic image is suitable for the dynamic analysis, the hardware processor displays, on the display, an analysis image that is obtained by analyzing a dynamic state of the dynamic image that is judged to be suitable.

13. A non-transitory recording medium storing a computer readable program causing a computer to perform:
acquiring that is acquiring a dynamic image obtained from dynamic imaging by radiation; and
judging that is performing a suitability judgement of the dynamic image for a dynamic analysis based on the dynamic image or an analysis result obtained by analyzing the dynamic image.

14. A non-transitory recording medium storing a computer readable program causing a computer to perform:
acquiring that is acquiring a dynamic image obtained from dynamic imaging by radiation;
outputting that is outputting unsuitability information when the dynamic image is unsuitable for a dynamic analysis; and
controlling that is controlling not to display, on a display, an analysis image obtained by analyzing a dynamic state of the dynamic image that is judged to be unsuitable.

15. A dynamic image processing method comprising:
acquiring a dynamic image that is obtained from dynamic imaging by radiation; and
performing a suitability judgement of the dynamic image for a dynamic analysis based on the dynamic image or an analysis result obtained by analyzing the dynamic image.

16. A dynamic image processing method comprising:
acquiring a dynamic image that is obtained from dynamic imaging by radiation;
outputting unsuitability information when the dynamic image is unsuitable for a dynamic analysis; and
controlling not to display, on a display, an analysis image obtained by analyzing a dynamic state of the dynamic image that is judged to be unsuitable.
